(19) United States
(12) Patent Application Publication
(10) Pub. No.: US 2014/0200548 A1
(43) Pub. Date: Jul. 17, 2014

(54) APPARATUSES AND KITS FOR GRINDING OR CUTTING SURGICAL FOAM AND METHODS RELATED THERETO

(71) Applicant: Merit Medical Systems, Inc., South Jordan, UT (US)

(72) Inventors: Fred Lampropoulos, Salt Lake City, UT (US); Gregory R. McArthur, Sandy, UT (US)

(73) Assignee: Merit Medical Systems, Inc., South Jordan, UT (US)

(21) Appl. No.: 14/157,296

(22) Filed: Jan. 16, 2014

Related U.S. Application Data
(60) Provisional application No. 61/753,730, filed on Jan. 17, 2013.

(51) Int. Cl. B02C 18/18; A61B 17/00; A61B 17/12; B26D 7/26; B26D 7/00

(52) U.S. Cl. CPC B02C 18/182; B26D 7/2628; B26D 7/0006; A61B 17/12109; A61B 17/0057
USPC 604/507; 241/169.1; 83/523; 83/613

(57) ABSTRACT

Apparatuses for grinding or cutting surgical foam are disclosed herein. Kits for grinding or cutting surgical foam are disclosed herein. Methods of grinding surgical foam with a plurality of teeth are disclosed herein. Methods of cutting surgical foam are disclosed herein. Methods of embolizing with ground or cut surgical foam are disclosed herein.

### CROSS-REFERENCE TO RELATED APPLICATIONS

Under 35 U.S.C. §119(e), this application claims the benefit of U.S. Provisional Patent Application No. 61/753,730, entitled “APPARATUSES AND KITS FOR GRINDING OR CUTTING SURGICAL FOAM AND METHODS RELATED THERETO,” filed Jan. 17, 2013, the contents of which are hereby incorporated herein by reference in their entirety.

### TECHNICAL FIELD

The present disclosure relates generally to medical devices. More specifically, the present disclosure relates to apparatuses and kits for grinding or cutting surgical foam and methods related thereto.

### BRIEF DESCRIPTION OF THE DRAWINGS

The embodiments disclosed herein will become more fully apparent from the following description and appended claims, taken in conjunction with the accompanying drawings. The drawings depict primarily generalized embodiments, which embodiments will be described with additional specificity and detail in connection with the drawings in which:

FIG. 1 illustrates a perspective view of one embodiment of an apparatus for grinding surgical foam.

FIG. 2 illustrates an exploded view of a sub-assembly of the embodiment of FIG. 1.

FIG. 3 illustrates an exploded view of a different sub-assembly of the embodiment of FIG. 1.

FIG. 4 illustrates a perspective view of a sub-assembly of the embodiment of FIG. 1.

FIG. 5 illustrates a perspective view of one housing component of the embodiment of FIG. 1.

FIG. 6 illustrates a view from underneath the embodiment of FIG. 1, but with a portion of the housing removed.

FIG. 7 illustrates a perspective view of one embodiment of a grinding disc.

FIG. 8 illustrates a front view of the grinding disc of FIG. 7.

FIG. 9 illustrates a cross-section of the embodiment of FIG. 1 along the line A-A.

FIG. 10 illustrates a perspective view of an embodiment of a gear useful in the embodiment of FIG. 1.

FIG. 11 illustrates a perspective view of an embodiment of another gear useful in the embodiment of FIG. 1.

FIG. 12 illustrates a perspective view of one embodiment of a rectangular piece of unground surgical foam.

FIG. 13 illustrates a perspective view of one embodiment of an apparatus for cutting surgical foam.

FIG. 14 illustrates a perspective view of one embodiment of an apparatus for cutting surgical foam.

### DETAILED DESCRIPTION

Surgical foams are used during surgery as a hemostatic to stop bleeding. Exemplary foams include SurgiFoam™ from Ethicon or Gelfoam™ from Pfizer. In some situations it is desirable to grind the surgical foam to a coarse powder, add saline and/or a contrast agent, and then inject the resulting slurry into a blood vessel to form an embolus in the blood vessel.

Apparatuses and kits for grinding surgical foams and methods related thereto are disclosed herein. It will be readily understood that the components of the embodiments as generally described below and illustrated in the Figures herein could be arranged and designed in a wide variety of different configurations. Thus, the following more detailed description of various embodiments, as described below and represented in the Figures, is not intended to limit the scope of the disclosure, but is merely representative of various embodiments. While the various aspects of the embodiments are presented in drawings, the drawings are not necessarily drawn to scale unless specifically indicated.

The phrases “operably connected to,” “connected to,” and “coupled to” refer to any form of interaction between two or more entities, including mechanical, electrical, magnetic, electromagnetic, fluid, and thermal interaction. Two entities may interact with each other even though they are not in direct contact with each other. For example, two entities may interact with each other through an intermediate entity.

In some embodiments of an apparatus for grinding surgical foam, the apparatus comprises a housing configured to receive an unground piece of surgical foam, such as a sheet. The housing is also configured to receive pulverized and shredded foam from a ground-up piece of surgical foam. The housing is also configured to allow removal of pulverized and shredded foam for use of the pulverized and shredded foam by a surgeon. The apparatus further comprises at least one shaft having a plurality of grinding discs attached to the shaft. Each grinding disc comprises a plurality of teeth. The plurality of grinding discs are spaced, located, and configured such that rotation of the plurality of grinding discs causes a piece of surgical foam in contact therewith to be pulled into the housing, while being pulverized and shredded. The plurality of grinding discs are operably connected to an actuator configured to rotate the plurality of grinding discs.

In some embodiments of a kit for grinding surgical foam, the kit comprises an apparatus for grinding surgical foam disclosed above. In some embodiments of a method of grinding surgical foam, the method comprises inserting a sheet of surgical foam into an apparatus configured to receive the sheet, such as the apparatus disclosed above, and actuating an actuator operably connected to a plurality of teeth so as to rotate the plurality of teeth until the sheet is pulverized and/or shredded.

In some embodiments of a method of embolization, the method of grinding surgical foam disclosed above is performed. After that, pulverized and shredded foam is removed from the apparatus and placed in a syringe. The pulverized and shredded foam is at least partially suspended in saline and/or contrast agent. The at least partially suspended foam is then injected from the syringe into a blood vessel to form an embolus in the blood vessel.

In some embodiments of an apparatus for cutting surgical foam, the apparatus comprises a rotary table configured to receive an uncut piece of surgical foam, such as a sheet of surgical foam. The rotary table is configured to be able to rotate the surgical foam 360 degrees along the plane of the rotary table. The apparatus further comprises a blade configured to cut the surgical foam. The apparatus further comprises positioning devices operably connected to the blade and configured to position the blade at any location on the rotary table. The apparatus further comprises an actuator operably coupled to the blade and the positioning devices, wherein operation of the actuator results in movement of the blade along at least one of the positioning devices.

In some embodiments of an apparatus for cutting surgical foam, the apparatus comprises a rail configured to lie upon an uncut piece of surgical foam, such as a sheet of surgical foam. The apparatus further comprises a blade configured to cut the surgical foam. The rail is operably coupled to a carrier operably coupled to the blade. The carrier is configured to slide along the rail. The apparatus further comprises an actuator operably coupled to the blade, such that operation of the actuator results in movement of the blade along the rail.

In some embodiments of a kit for cutting surgical foam, the kit comprises an apparatus for cutting surgical foam disclosed above. In some embodiments of a method of cutting surgical foam, the method comprises placing a piece of surgical foam on or under an apparatus for cutting surgical foam disclosed above and actuating the actuator until the surgical foam is cut. The methods may further comprise selecting a specific width of surgical foam desired and adjusting the apparatus accordingly.

In some embodiments of a method of treating bleeding, the method of cutting surgical foam disclosed above is performed. After that, cut foam is removed from the apparatus and rolled into a torpedo shape. Rolled foam is placed in a syringe. Saline and/or contrast agent is added to the syringe. The saline and/or contrast agent and rolled foam are agitated until at least a portion of the rolled foam is suspended in the saline and/or contrast agent. The at least partially suspended foam is then extruded from the syringe at a wound site that is bleeding to attempt to stop the bleeding.

In some embodiments of a method of embolization, the method of cutting surgical foam disclosed above is performed. After that, cut foam is removed from the apparatus and rolled into a torpedo shape. Rolled foam is placed in a syringe. Saline and/or contrast agent is added to the syringe. The saline and/or contrast agent and rolled foam are agitated until at least a portion of the rolled foam is suspended in the saline and/or contrast agent. The at least partially suspended foam is then injected from the syringe into a blood vessel to form an embolus in the blood vessel.

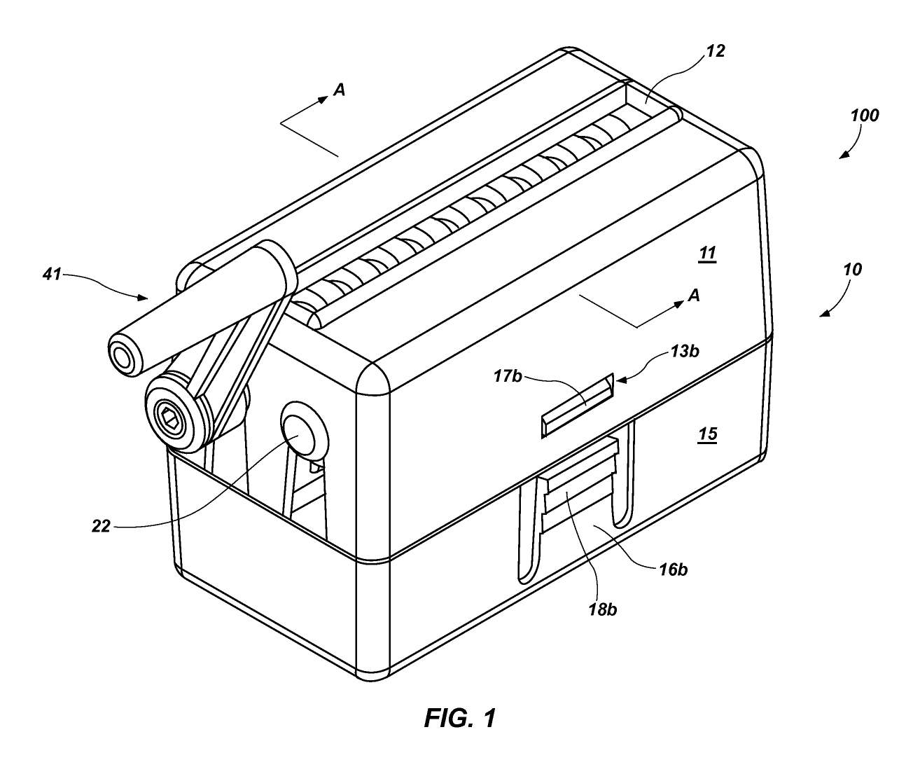

FIG. 1 illustrates one embodiment of an apparatus 100 for grinding surgical foam. The apparatus 100 of FIG. 1 comprises various components and materials as further detailed below. Additionally, any combination of the individual components may comprise an assembly or subassembly for use in pulverizing and shredding surgical foam.

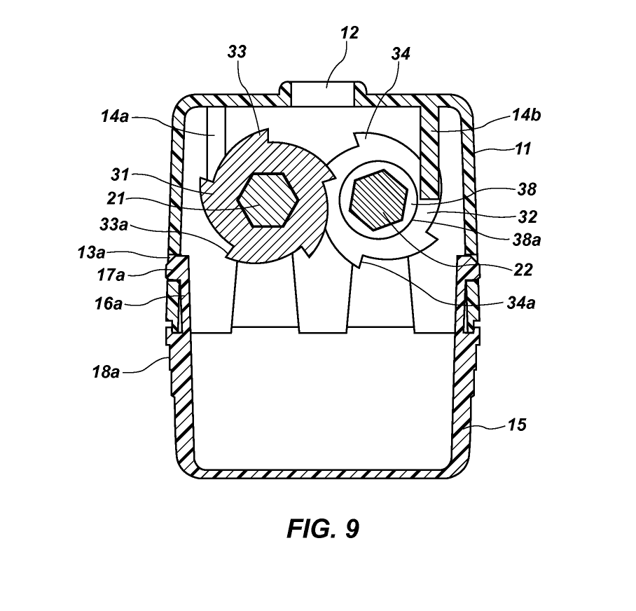

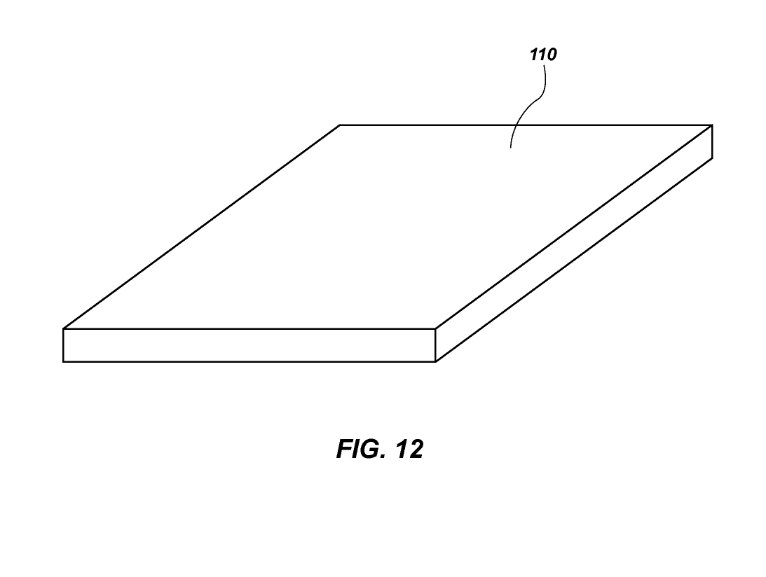

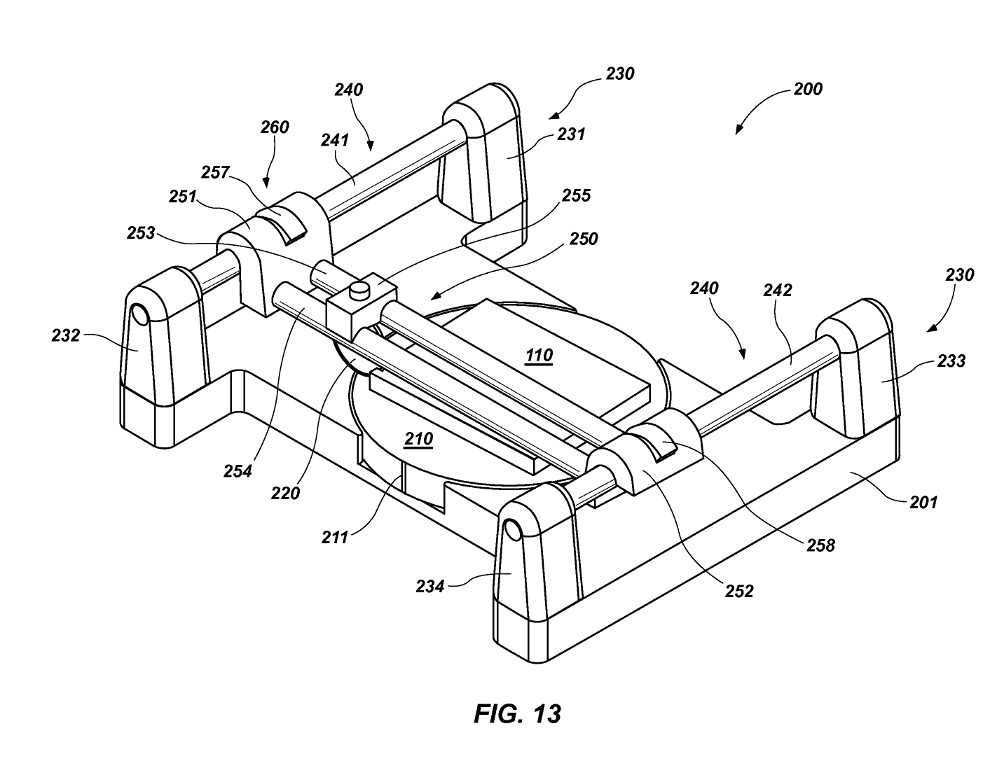

Apparatus 100 includes housing 10. Housing 10 comprises top cover 11 configured to receive sheet 110 of unground surgical foam (see FIG. 12). Top cover 11 includes mouth 12 configured to allow insertion of sheet 110 inside top cover 11. Housing 10 also comprises catch cup 15 configured to receive pulverized and shredded foam from a ground-up sheet 110. Catch cup 15 is configured to allow removal of pulverized and shredded foam for use of the pulverized and shredded foam by a surgeon. Catch cup 15 comprises depressible tab 16a (shown in FIG. 9) and depressible tab 16b on opposing sides of catch cup 15. The ends of depressible tabs 16a and 16b each include latches 17a (shown in FIG. 9) and 17b, respectively. Latches 17a (shown in FIG. 9) and 17b are configured to mate with receivers 13a and 13b formed in opposing sides of top cover 11. Latches 17a and 17b and receivers 13a and 13b are configured so that when latch 17a is mated with receiver 13a and latch 17b is mated with receiver 13b, then catch cup 15 is secured to top cover 11. Depressible tabs 16a and 16b each include grips 18a and 18b, respectively, formed on the outside thereof. Grips 18a and 18b assist in depressing depressible tabs 16a and 16b to release catch cup 15 from top cover 11.

Depressible tabs 16a and 16b may be configured so that an individual may hold apparatus 100 in a first hand, depress depressible tabs 16a and 16b with the thumb and second finger of a second hand, and remove catch cup 15 from top cover 11 with the second hand while still holding top cover 11 with the first hand. The individual may then remove pulverized and shredded foam from catch cup 15, such as by pouring the pulverized and shredded foam into an open syringe. The individual may use a funnel when pouring the pulverized and shredded foam into the syringe.

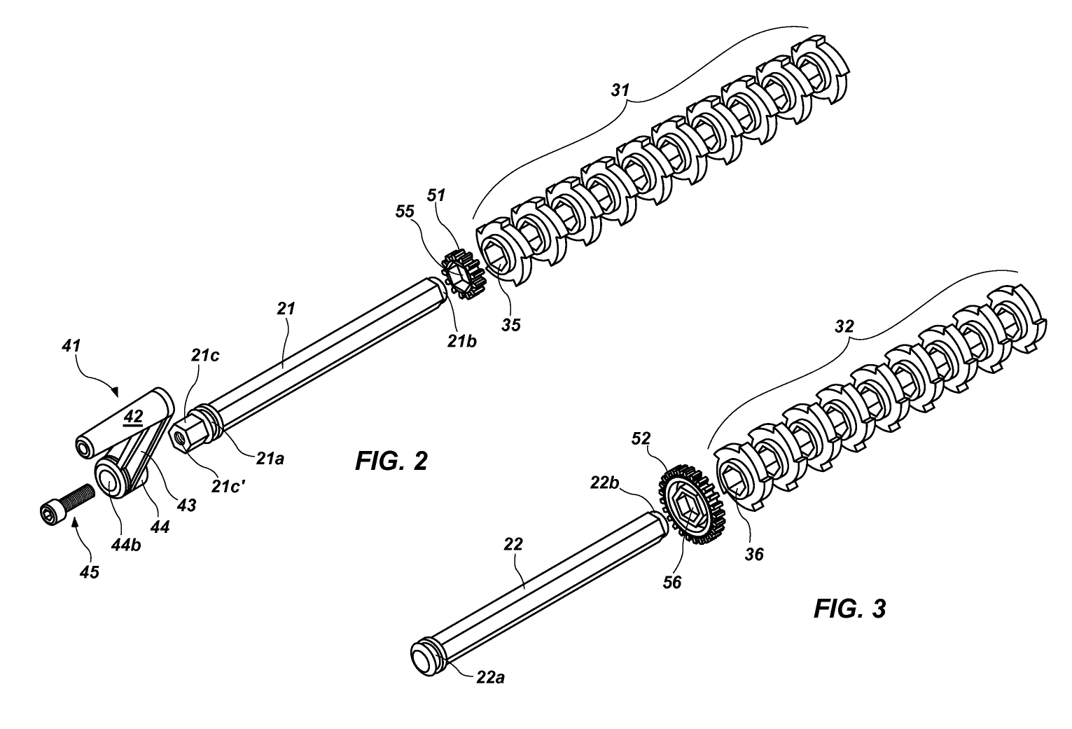

Referring collectively to FIGS. 2, 3 and 4, apparatus 100 may further comprise a shaft 21 having a plurality of grinding discs 31 attached thereto. Apparatus 100 may further comprise a shaft 22 having a plurality of grinding discs 32 attached thereto. Grinding discs 31 and 32 may be mirror images of each other. Each of grinding discs 31 and 32 comprise a plurality of teeth 33 and 34, respectively.

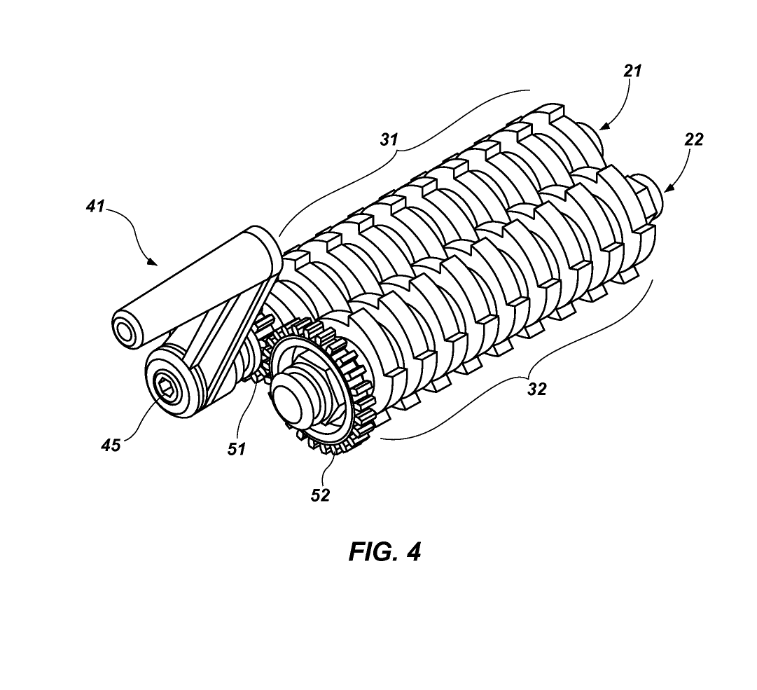

FIG. 2 illustrates an exploded view of an assembled shaft 21. FIG. 3 illustrates an exploded view of an assembled shaft 22. FIG. 4 illustrates assembled shafts 21 and 22 intermeshing. Gear 51 is operably connected to shaft 21. Gear 52 is operably connected to Shaft 22. Gear 51 is meshed with gear 52 so that when shaft 21 is rotated clockwise, shaft 22 is rotated counter-clockwise. Shaft 21 is operably connected to hand crank 41.

The proximal end of shaft 21 further comprises hollow shank 21c. “Proximal” as used herein refers to near the end of apparatus 100 where hand crank 41 is attached. Hollow shank 21c extends beyond the outside of top cover 11 and has a hexagonal outer surface. The hexagonal outer surface of hollow shank 21c is configured to mate with the hexagonal inner surface (not shown) of socket head 44 of hand crank 41, so that rotation of hand crank 41 results in rotation of shaft 21. Hand crank 41 also comprises handle 42 connected to lever arm 43, which in turn is connected to socket head 44.

Hollow shank 21c also comprises threaded opening 21c′ configured to receive cap screw 45. Socket head 44 has a recessed annulus 44b that opens into the hexagonal inner surface of socket head 44. The screw portion of cap screw 45 is configured to insert through the opening of recessed annulus 44b and screw into threaded opening 21c″. Screwing cap screw 45 into threaded opening 21c″ pulls the head of cap screw 45 against the surfaces of recessed annulus 44b, to thereby secure hand crank 41 to hollow shank 21c.

In one embodiment, shafts 21 and 22 are essentially hexagonal cylinders. Grinding discs 31 and 32 include hexagonal holes 35 and 37, respectively. Shaft 21 is configured to slide through hexagonal holes 35 of ten grinding discs 31. The outer surface of shaft 21 mates with the inner surface of hexagonal holes 35 so that when shaft 21 is rotated, then grinding discs 31 are also rotated. Likewise, shaft 22 is configured to slide through hexagonal holes 36 of nine grinding discs 32. The outer surface of shaft 22 mates with the inner surface of hexagonal holes 36 so that when shaft 22 is rotated, then grinding discs 32 are also rotated. Gear 51 also includes a hexagonal hole 55 to allow shaft 21 to slide through and engage with hexagonal hole 55. Likewise, Gear 52 also includes a hexagonal hole 56 to allow shaft 22 to slide through and engage with hexagonal hole 56.

Hand crank 41 is configured for operation by a single individual, such as member of a surgical team. Clockwise rotation of hand crank 41 results in clockwise rotation of shaft 21 which results in clockwise rotation of grinding discs 31. Clockwise rotation of shaft 21 results in counter-clockwise rotation of shaft 22, which results in counter-clockwise rotation of grinding discs 32. Grinding discs 31 and 32 are spaced, located, and configured such that clockwise rotation of grinding discs 31 and counter-clockwise rotation of grinding discs 32 causes sheet 110 inserted in mouth 12 and in contact with grinding discs 31 and 32 to be pulled into housing 10, while being pulverized and shredded by grinding discs 31 and 32.

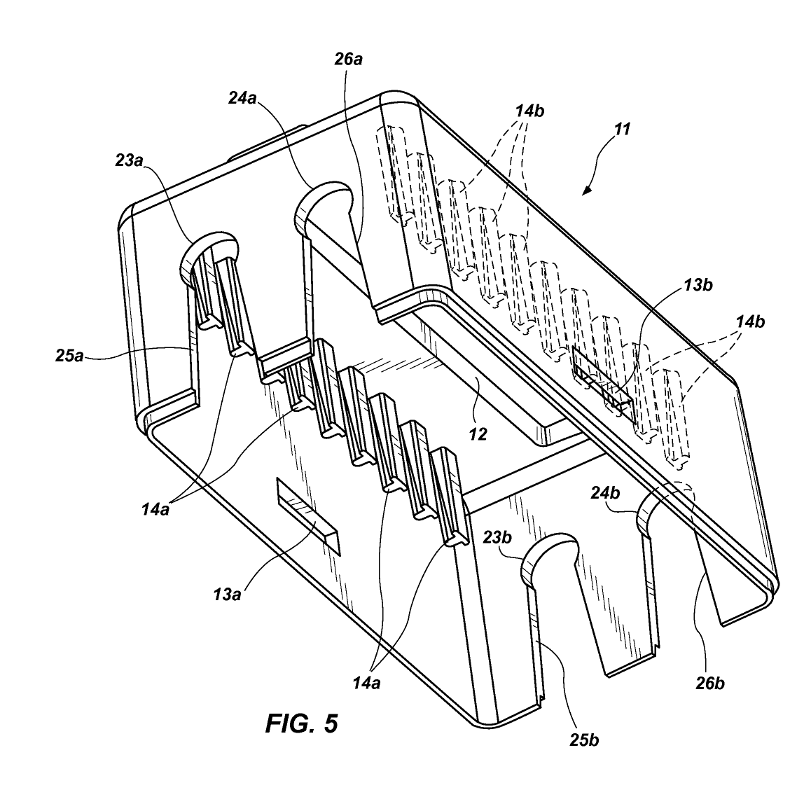

FIG. 5 illustrates a perspective view of top cover 11 from beneath top cover 11. Top cover 11 includes hub 23a, hub 23b, hub 24a, and hub 24b. Top cover 11 further includes grooves 25a and 25b configured to allow circular ring 21a and circular region 21b to be snapped into hubs 23a and 23b, respectively. Likewise, top cover 11 further includes grooves 26a and 26b configured to allow circular ring 22a and circular region 22b to be snapped into hubs 24a and 24b, respectively. Top cover 11 is made of a material sufficiently elastic so that top cover 11 can deform while shafts 21 and 22 are pressed into hubs 23a and 23b and 24a and 24b, respectively, and then return to the original shape of top cover 11 after shafts 21 and 22 are in place.

The proximal end of shaft 21 comprises a circular ring 21a configured to mate with hub 23a. The distal end of shaft 21 comprises a circular region 21b configured to mate with hub 23b. Likewise, the proximal end of shaft 22 comprises a circular ring 22a configured to mate with hub 24a. The distal end of shaft 22 comprises a circular region 22b configured to mate with hub 24b.

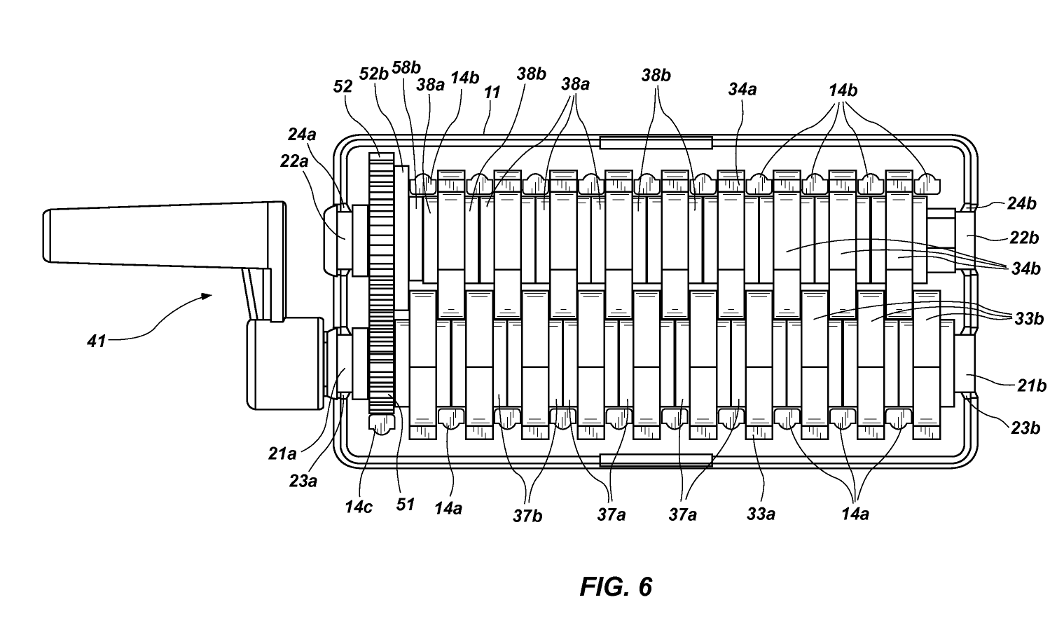

FIG. 6 illustrates a view from underneath a fully-assembled apparatus 100, but with catch cup 15 removed. When circular ring 21a is mated with hub 23a and circular region 21b is mated with hub 23b, shaft 21 is supported by top cover 11 and allowed to rotate along the surfaces of hubs 23a and 23b. Likewise, when circular ring 22a is mated with hub 24a and circular region 22b is mated with hub 24b, shaft 22 is supported by top cover 11 and allowed to rotate along the surfaces of hubs 24a and 24b. Nine grinding discs 32 are placed adjacent to each other on shaft 22. Ten grinding discs 31 are placed adjacent to each other on shaft 21.

Pillars 14a, 14b, and 14c extend from the underside of top cover 11. Nine pillars 14a are interstitially-spaced between adjacent grinding discs 31. Eight pillars 14b are interstitially-spaced between adjacent grinding discs 32. One pillar 14b is placed behind the distal face of the distal-most grinding disc 32. One pillar 14b is placed between the proximal face of the proximal most grinding disc 32 and the distal face of gear 52. Pillar 14c is placed in close proximity to the teeth of gear 51 to prevent shredded foam from becoming entrained in the gear teeth of gear 51.

In one embodiment, pillars 14a and 14b are mirror images of each other. Pillars 14a are located in close proximity to radial edges 37b′ and 37a′ of adjacent circular ledges 37b and 37a. Pillars 14b are located in close proximity to radial edges 38b′ and 38a′ of adjacent circular ledges 38b and 38a. Pillars 14a and 14b prevent chunks of shredded foam from recirculating with teeth 33 and 34 as grinding discs 31 and 32 complete full revolutions. Pillars 14a and 14b help to keep chunks of shredded foam in catch cup 15.

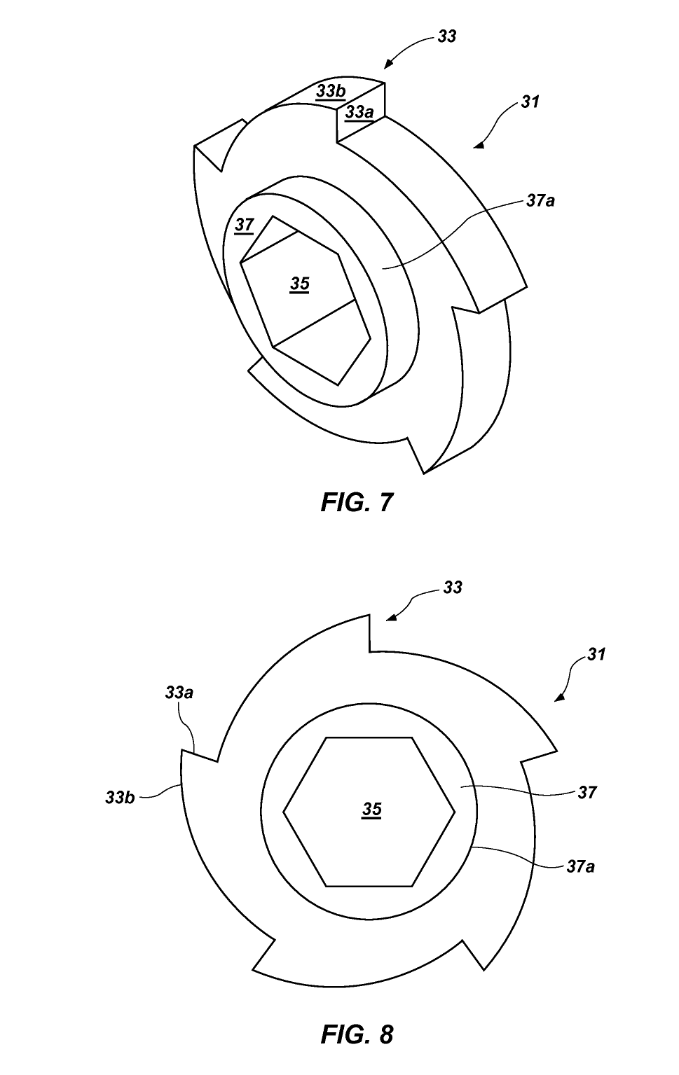

FIG. 7 illustrates a perspective view of one grinding disc 31. FIG. 8 illustrates a front view of one grinding disc 31. As noted previously, grinding disc 31 includes teeth 33 and hexagonal hole 35. Grinding disc 31 includes five teeth 33. Each of teeth 33 is fin-shaped and includes a biting surface 33a and an arced surface 33b. Each biting surface 33a is a flat surface that extends radially from the axial center of grinding disc 31. Each arced surface 33b extends from the outward most edge of a biting surface 33a to the inward most edge of an adjacent biting surface 33a, giving grinding disc 31 the appearance of a spiral star.

Each grinding disc 31 includes a circular ledge 37a that protrudes axially outward (relative to shaft 21) from the center of the proximal face of grinding disc 31 (relative to hand crank 41) surrounding hexagonal hole 35. Each grinding disc 31 includes a circular ledge 37b that protrudes axially outward (relative to shaft 21) from the center of the distal face of grinding disc 31 (relative to hand crank 41) surrounding hexagonal hole 35. The thickness of each of circular ledges 37a and 37b is about one-half the width of teeth 33, such that when two grinding discs 31 are next to each other on shaft 21, the width of circular ledge 37b of the first grinding disc 31 and the width of circular ledge 37a of the second grinding disc 31 combined together is slightly greater than the width of teeth 33. Circular ledges 37a and 37b and corresponding circular ledges 38a and 38b are sufficiently thick that teeth 33 and teeth 34 do not interfere with each other as grinding discs 31 and 32 are rotating. The radial edge of circular ledges 37a and 37b is about length of a biting surface 33a away from the bottom of a biting surface 33a, sufficient that circular ledges 37a and 37b do not interfere with teeth 34 of grinding discs 32. Likewise, for the radial edges of circular ledges 38a and 38b of grinding discs 32

FIG. 9 illustrates a cross-section of apparatus 100 along the line A-A and illustrates the interplay of grinding discs 31 and 32 and pillars 14. As mentioned above, grinding discs 32 may be mirror-images to grinding discs 31. Grinding discs 32 may be identical to grinding discs 31, but a mirror image because the orientation of grinding discs 32 is the reverse of grinding discs 31. FIG. 9 illustrates on the right side a proximal view of a grinding disc 32 with a circular ledge 38a. FIG. 9 illustrates on the left side a cross-sectional slice along the length of a grinding disc 31 as it overlaps the proximal face of a grinding disc 32.

Biting surfaces 33a and 34a of teeth 33 and 34, respectively, are configured to engage with a sheet 110 and pulverize and/or shred a portion of sheet 110. Additionally, as biting surfaces 33a and 34a engage sheet 110, force is exerted on sheet 110 that tends to draw sheet 110 through mouth 12 and into additional engagement with grinding discs 31 and 32.

Each of teeth 33 of the single grinding disc 31 are configured so that the outward most edge of each biting surface 33a rotates in close proximity to the radial edges of circular ledge 38a. Likewise, each of teeth 34 of the single grinding disc 32 are configured so that the outward most edge of each biting surface 34a rotates in close proximity to the radial edge of circular ledge 37b (not shown in FIG. 9). Although not shown in FIG. 9, it should be understood that each of teeth 34 of the single grinding disc 32 also rotates in close proximity to the radial edge of the circular ledge 37a of the grinding disc 31 that is immediately behind the illustrated cross-sectionally sliced grinding disc 31. Likewise, it should be understood that each of teeth 33 of the cross-sectionally sliced grinding disc 31 also rotate in close in close proximity to the radial edge of the circular ledge 38b of the grinding disc 32 immediately in front of the illustrated grinding disc 32.

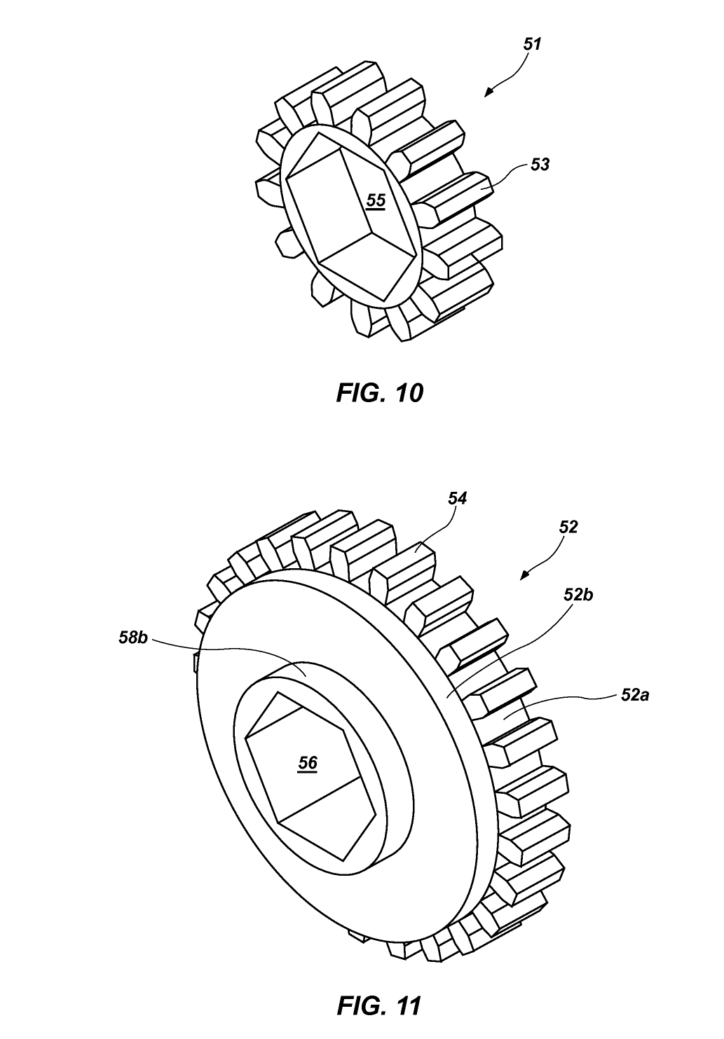

FIG. 10 illustrates a perspective view of gear 51. Gear 51 has a smaller circumference than gear 52, which results in shaft 21 rotating about twice as many times as shaft 22 (i.e., gear 52 and gear 51 have a two-to-one gear ratio). This may have the benefit of grinding discs 31 on shaft 21 generally pulverizing sheet 110 while the grinding discs 32 on shaft 22 generally shred sheet 110. Gear 51 and gear 52 may designed for any number of ratios depending upon a desired relative shaft speed of shaft 21 to shaft 22. For example, gear 51 and gear 52 may have a one-to-one gear ratio so that shaft 22 turns at the same speed as shaft 21.

FIG. 11 illustrates a perspective view of gear 52 from the distal side. The gear teeth 54 of gear 52 are the same width as gear teeth 53 of gear 51. However, gear edge 52a of gear 52 includes region 52b distal to gear teeth 54 about equal in width to a circular ledge 38a or 38b of a grinding disc 32 where gear teeth 54 are not present. Additionally, a circular ledge 58b protrudes axially outward (relative to shaft 22) from the center of the distal face of gear 52, surrounding hexagonal hole 56. The thickness of circular ledge 58b is about one-half the width of teeth 34, which is the same as about the width of region 52b. As illustrated in FIG. 6, circular ledge 58b is configured to function as a circular ledge 38b of a grinding disc 32 and mate with an adjacent circular ledge 38a. As also illustrated in FIG. 6, region 52b and the distal face of gear 52 provide an interstitial boundary for the proximal most grinding disk 31 on shaft 21.

FIG. 12 illustrates one embodiment of a sheet 110 of a rectangular piece of unground surgical foam.

It should be understood that embodiments of an apparatus for grinding surgical foam include variations of apparatus 100. For example, regarding housing 10, mouth 12 may be configured to receive surgical foam shapes other than a rectangular sheet. Additionally, the shape of housing 10 may be configured to accommodate variations in the number and diameter of grinding discs 31 and 32. Instead of depressible tabs 16a and 16b, other means for detaching catch cup 15 may be used. For example, catch cup 15 may be circular in shape and be threaded onto top cover 11. Furthermore, instead of catch cup 15 being removable, a door or port may be present in housing 10 to allow removal of pulverized and shredded foam. Likewise, catch cup 15 may be hinged onto top cover 11 and capable of swinging open to allow removal of pulverized and shredded foam.

In other variations of apparatus 100, shafts 21 and 22 are not primarily hexagonal cylinders, but may be any shape compatible with rotation of grinding discs 31 and 32. For example, shafts 21 and 22 may be splined cylinders or cylinders with a keyseat groove along the length of the shaft. The hexagonal holes 35 and 36 of grinding disc 31 and 32, respectively, may be configured as necessary to mate with the configuration of shafts 21 and 22.

Likewise, grinding discs 31 and 32 may use some other means other than circular ledges 37a, 37b, 38a, and 38b for governing the meshing of teeth 33 and teeth 34. For example, circular ledges 37a, 37b, 38a, and 38b may not be present and the coupling of grinding discs 31 and 32 to shafts 21 and 22, respectively, may govern the meshing of teeth 33 and 34.

Additionally, grinding discs 31 and 32 may be unitary with shafts 21 and 22, respectively. For example, shaft 21 may comprise a plurality of teeth 33 and shaft 22 may comprise a plurality of teeth 34, where the plurality of teeth 33 and 34 are spaced, located, and configured such that rotation of the plurality of teeth causes a piece of surgical foam in contact therewith to be pulled into housing 10 while being pulverized and shredded. Likewise, grinding discs 31 and 32 may include any number of teeth 33 and 34, respectively. Teeth 33 and 34 may be any shape compatible with pulverizing and/or shredding surgical foam.

Likewise, gears 51 and 52 may be unitary with shafts 21 and 22, respectively. As discussed above, gears 51 and 52 may be at any gear ratio desired. Alternatively, instead of shaft 22 coupled to shaft 21 via gears 51 and 52, shaft 22 may be operably coupled to shaft 21 in any manner that provides for rotation of shaft 22 when shaft 21 is rotated. For example, shaft 22 may be coupled to shaft 21 via a belt drive.

In other variations, apparatus 100 may be actuated by any number of means. For example, hand crank 41 may be one continuous piece with shaft 21 or may be connected to shaft 21 by glue, ultrasonic welding, or via snap-fit or press-fit connections. Additionally, instead of hand crank 41 being directly coupled to shaft 21, hand crank 41 may rotate a shaft that in turn rotates shaft 21 and/or shaft 22. Instead of hand crank 41 being driven by rotary movement, hand crank 41 may be configured for linear movement which is then translated into rotary movement of shafts 21 and 22.

In other variations, hubs 23a, 23b, 24a, and 24b and grooves 25a, 25b, 26a, and 26b may be modified or may not be present and shafts 21 and 22 may be attached to housing 10 in some other manner. For example, hubs 23b and 24b may be recessed cavities for receiving circular regions 21b and 22b of shafts 21 and 22, respectively. In another example, hubs 23a and 24a may be complete circles and circular rings 21a and 22a of shafts 21 and 22, respectively, may be designed to snap-fit into place with hubs 23a and 24a.

In still other variations, pillars 14a and 14b may be configured and located in any manner compatible with preventing shredded foam from recirculating within housing 10. For example, instead of a pillar shape, a guard could be present on each long side of the inner surface of top cover 11. An edge of each guard could mirror the profile of the plurality of grinding discs 31 and 32, respectively, and prevent shredded foam from recirculating.

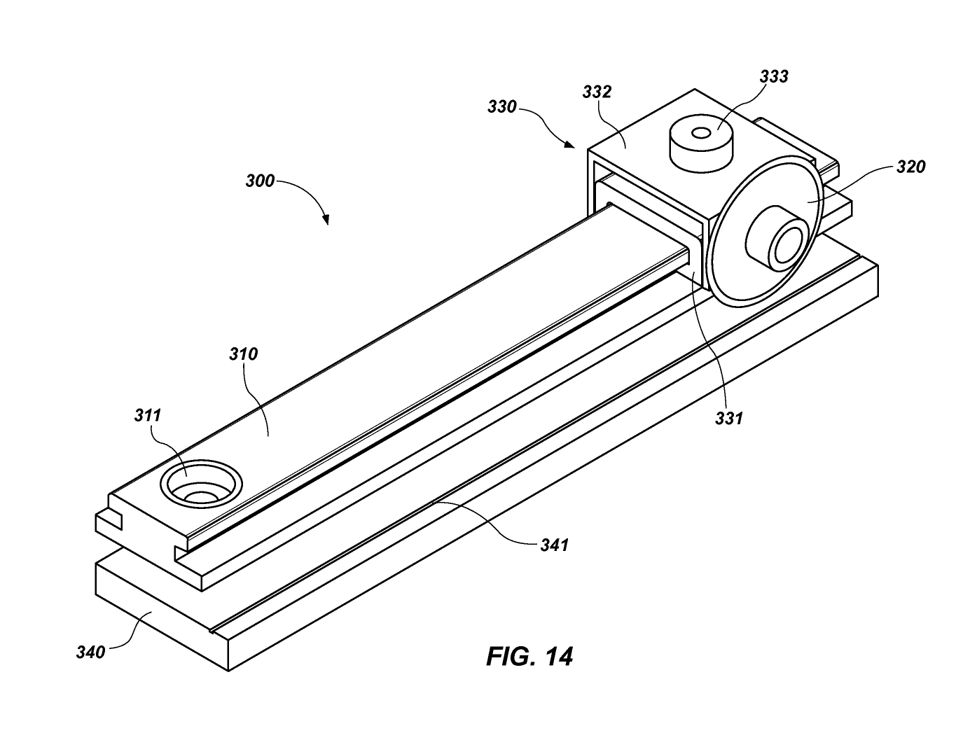

FIG. 13 illustrates one embodiment of an apparatus 200 for cutting surgical foam. The apparatus 200 of FIG. 13 comprises various components and materials as further detailed below. Additionally, any combination of the individual components may comprise an assembly or subassembly for use in cutting surgical foam.

Apparatus 200 comprises a rotary table 210 configured to receive an uncut piece of sheet 110 of surgical foam. Rotary table 210 is configured to be able to rotate sheet 110 360 degrees along the plane of rotary table 210. Apparatus 200 further comprises a blade 220 configured to cut sheet 110. Apparatus 200 further comprises positioning devices 260 operably connected to blade 220 and configured to position blade 220 at any location on rotary table 210. The apparatus further comprises an actuator (not shown) operably coupled to blade 220 and positioning devices 260, wherein operation of the actuator results in movement of blade 220 along at least one of positioning devices 260.

Apparatus 200 further comprises base 201 configured to support rotary table 210. Rotary table 210 comprises a plurality of positioning grooves 211. Base 201 comprises at least one releasable detent (not shown) configured for releasably engaging positioning groove 211 and thereby securing the rotary position of rotary table 210 until the at least one releasable detent is released. Sheet 110 may be secured to rotary table 210 with an adhesive. In other embodiments, sheet 110 may be retained within a recessed portion of rotary table 210 corresponding to the size of sheet 110.

Positioning members 260 comprise supports 230, rods 240, and rack 250. Supports 230 comprise supports 231, 232, 233, and 234. Rods 240 comprise rod 241 and rod 242. Rack 250 comprises slide 251, slide 252, gear rod 253, gear rod 254, and hanger 255.

Supports 231 and 232 attach to base 201 and attach to rod 241. Supports 233 and 234 attach to base 201 and attach to rod 242. Supports 230 prevent rods 240 from moving as rack 250 slides along rods 240.

Slide 251 slidably engages rod 241. Slide 252 slidably engages rod 242. Slides 251 and 252 each include a clamp 257 and 258, respectively, for locking the position of rack 250 on rods 240.

Hanger 255 is operably connected to blade 220 and the actuator. Hanger 255 is slidably engaged with the upper surfaces of gear rods 253 and 254. Gear rods 253 and 254 include a gear track (not shown) on the underside of each of gear rods 253 and 254. The gear track runs the length of gear rods 253 and 254.

The actuator is operably coupled to the gear track and blade 220. The actuator includes driving gears for mating with the gear tracks of gear rods 253 and 254. Actuation of the driving gears causes hanger 255 to travel forwards or backwards along gear rods 253 and 254 and thereby move the position of blade 220. The actuator may include an additional gear operably coupled to blade 220. Forward actuation of the driving gears may result in counter-clockwise rotation of blade 220, from the perspective of FIG. 13. Backward actuation of the driving gears results in clockwise rotation of blade 220. The actuator may comprise a hand crank for operating the actuator.

It should be understood that embodiments of an apparatus for cutting surgical foam include variations of apparatus 200. For example, blade 220 may be a straight edge and the actuator may not be configured for rotating blade 220.

FIG. 14 illustrates one embodiment of an apparatus 300 for cutting surgical foam. The apparatus 300 of FIG. 14 comprises various components and materials as further detailed below. Additionally, any combination of the individual components may comprise an assembly or subassembly for use in cutting surgical foam.

Apparatus 300 comprises a rail 310 configured to lie upon an uncut piece of surgical foam, such as a sheet 110 of surgical foam. Apparatus 300 further comprises a blade 320 configured to cut the surgical foam. Rail 310 is operably coupled to a carrier 330 operably coupled to blade 320. Carrier 330 is configured to slide along rail 310. Apparatus 300 further comprises an actuator (not shown) operably coupled to blade 320. Operation of the actuator results in movement of blade 320 along rail 310.

Apparatus 300 further comprises backboard 340. Backboard 340 and rail 310 sandwich sheet 110 (not shown) of surgical foam. Backboard 340 includes groove 341 for receiving blade 320 as a sheet 110 is cut. Backboard 340 may be operable connected to rail 310.

Carrier 330 comprises a runner 331 configured to slidably engage with rail 310. Rail 310 includes lateral grooves on either side of rail 310. Runner 331 includes clasps for engaging with the grooves of rail 310 while allowing carrier 330 to slide along rail 310. Carrier 330 further comprises blade holder 332 configured to mate with runner 331. Blade holder 332 is also configured to allow rotation of blade 320 by the actuator. Blade holder 332 is operably connected to the actuator.

Backboard 340 further comprises a gear track (not shown) that runs the length of backboard 340 parallel to groove 341. The actuator is operably coupled to the gear track and blade 320. The actuator includes driving gears for mating with the gear track. Actuation of the driving gears causes carrier 330 to travel forwards or backwards along rail 310 and thereby move the position of blade 320. The actuator includes an additional gear operably coupled to blade 320. Forward actuation of the driving gears results in clockwise rotation of blade 320, from the perspective of FIG. 14. Backward actuation of the driving gears results in counter-clockwise rotation of blade 320. The actuator may comprise a hand crank for operating the actuator.

Rail 310 further comprises safety stop 311 in one end of the upper surface of rail 310. A corresponding safety stop (not shown) is at the other end of the upper surface of rail 310. Safety stop 311 may comprise a cylindrical recess in the upper surface of rail 310. Carrier 330 comprises safety 333. Safety 333 comprises a cylinder with an upper half and a lower half, wherein the lower half sits inside the upper half. A spring is located inside the upper half and the lower half of safety 333. When safety 333 reaches safety stop 311, the spring inside safety 333 is able to decompress and the lower half of safety 333 is pushed into safety stop 311. The bottom surface of safety stop 311 may be pushed upwards from underneath rail 310. Safety 333 may be released by pushing the bottom surface of safety stop 311 upwards and sufficiently compressing the spring inside safety 333 so that carrier 330 may be moved away from safety stop 311.

It should be understood that embodiments of an apparatus for cutting surgical foam include variations of apparatus 300. For example, blade 320 may be a straight edge and the actuator may not be configured for rotating blade 320.

Additionally, it should be understood that any of the connections of any of the components of apparatus 100, apparatus 200, and apparatus 300 may connected in any manner suitable for performing the intended function.

Apparatus 100, apparatus 200, and apparatus 300 may be made from a variety of materials, including various plastics. Apparatus 100, apparatus 200, and apparatus 300 may be configured to be sufficiently lightweight that a single individual, such as a surgeon, may be able to hold the apparatus in one hand. Additionally, apparatus 100, apparatus 200, and apparatus 300 may be made of materials that are at least partially biodegradable. Apparatus 100, apparatus 200, and apparatus 300 may be intended to be disposable after a single use or several uses in a single surgical procedure.

In some embodiments of a kit for grinding surgical foam, the kit comprises apparatus 100. The kit may further comprise sheet 110 of surgical foam.

In some embodiments of a method of grinding surgical foam, the method comprises inserting sheet 110 of surgical foam into mouth 12 of apparatus 100 and actuating hand crank 41 until sheet 110 is pulverized and shredded. The method may further comprise performing steps during a surgery. The method may further comprise disposing of apparatus 100 after the surgery is completed.

In some embodiments of a method of treating bleeding, the method of grinding surgical foam with apparatus 100 is performed. After that, pulverized and shredded foam is removed from apparatus 100 and placed in a syringe. The pulverized and shredded foam is at least partially suspended with saline and/or a contrast agent. The at least partially suspended foam is then extruded from the syringe at a wound site that is bleeding to attempt to stop the bleeding.

In some embodiments of a method of embolization, the method of grinding surgical foam with apparatus 100 is performed. After that, pulverized and shredded foam is removed from apparatus 100 and placed in a syringe. The pulverized and shredded foam is at least partially suspended with saline and/or a contrast agent. The at least partially suspended foam is then injected from the syringe into a blood vessel to form an embolus in the blood vessel.

In some embodiments of a kit for cutting surgical foam, the kit comprises either an apparatus 200 or an apparatus 300 for cutting surgical foam. The kit may further comprise a sheet 110 of surgical foam.

In some embodiments of a method of cutting surgical foam, the method comprises applying an adhesive to rotary table 210 of apparatus 200, placing sheet 110 of surgical foam on the adhesive, and actuating the actuator until sheet 110 is cut. The methods may further comprise selecting a specific shape of surgical foam desired and adjusting the starting position of blade 220 accordingly.

In some embodiments of a method of cutting surgical foam, the method comprises placing sheet 110 of surgical foam under apparatus 300 and actuating the actuator until sheet 110 is cut. The methods may further comprise selecting a specific width of surgical foam desired and adjusting portion of sheet 110 extending beyond groove 341 accordingly.

In some embodiments of a method of treating bleeding, the method of cutting surgical foam with either apparatus 200 or apparatus 300 is performed. After that, cut foam is removed from the apparatus and rolled into a torpedo shape. Rolled foam is placed in a syringe. Saline and/or a contrast agent is added to the syringe. The saline and/or contrast agent and rolled foam are agitated until at least a portion of the rolled foam is suspended in the saline and/or contrast agent. The at least partially suspended foam is then extruded from the syringe at a wound site that is bleeding to attempt to stop the bleeding.

In some embodiments of a method of embolization, the method of cutting surgical foam with either apparatus 200 or apparatus 300 is performed. After that, cut foam is removed from the apparatus and rolled into a torpedo shape. Rolled foam is placed in a syringe. Saline and/or a contrast agent is added to the syringe. The saline and/or contrast agent and rolled foam are agitated until at least a portion of the rolled foam is suspended in the saline and/or contrast agent. The at least partially suspended foam is then injected from the syringe into a blood vessel to form an embolus in the blood vessel.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

1. An apparatus for grinding surgical foam, the apparatus comprising:
a housing configured to receive an unground piece of surgical foam, wherein the housing is configured to receive pulverized and shredded foam from a ground-up piece of the surgical foam and wherein the housing is also configured to allow removal of pulverized and shredded foam for use of the pulverized and shredded foam by a surgeon;
at least one shaft having a plurality of teeth attached thereto, wherein the plurality of teeth are spaced, located, and configured such that rotation of the plurality of teeth causes a piece of surgical foam in contact therewith to be pulled into the housing, while being pulverized and shredded; and
an actuator operably connected to the plurality of teeth and configured to rotate the plurality of teeth.

2. The apparatus of claim 1, further comprising two shafts operably connected to each other and having a plurality of teeth attached thereto.

3. The apparatus of claim 1, wherein each shaft present comprises a plurality of grinding discs attached to each shaft, wherein each grinding disc comprises a plurality of teeth, wherein the plurality of grinding discs are spaced, located, and configured such that rotation of the plurality of grinding discs causes a piece of surgical foam in contact therewith to be pulled into the housing, while being pulverized and shredded.

4. The apparatus of claim 3, wherein the plurality of grinding discs are operably connected to an actuator configured to rotate the plurality of grinding discs.

5. The apparatus of claim 4, comprising a first shaft and a second shaft, wherein the first shaft is coupled to the actuator and the second shaft is operably connected to the first shaft.

6. The apparatus of claim 5, wherein the first shaft comprises a first gear and wherein the second shaft comprises a second gear, wherein the first gear is configured to mesh with the second gear.

7. The apparatus of claim 1, further comprising a removable catch cup, wherein the catch cup is configured to receive pulverized and shredded foam and allow removal the pulverized and shredded foam.

8. The apparatus of claim 7, wherein the apparatus may be held in one hand and the catch cup removed by a second hand.

9. The apparatus of claim 1, wherein the actuator comprises a hand crank.

10. The apparatus of claim 1, further comprising recirculation preventors configured to prevent pulverized and shredded surgical foam from recirculating within the apparatus.

11. An apparatus for cutting surgical foam, the apparatus comprising:
a rotary table configured to receive an uncut piece of surgical foam, wherein the rotary table is configured to be able to rotate the surgical foam 360 degrees along the plane of the rotary table;
a blade configured to cut the surgical foam;
positioning devices operably connected to the blade and configured to position the blade at any location on the rotary table;
an actuator operably coupled to the blade and the positioning devices, wherein operation of the actuator results in movement of the blade along at least one of the positioning devices.

12. An apparatus for cutting surgical foam, the apparatus comprising:
a rail configured to lay upon an uncut piece of surgical foam;
a blade configured to cut the surgical foam;
a carrier operably coupled to the rail and operably coupled to the blade, wherein the carrier is configured to slide along the rail; and
an actuator operably coupled to the blade, wherein operation of the actuator results in movement of the blade along the rail.

13. A kit for grinding surgical foam, the kit comprising:
an apparatus, comprising:
a housing configured to receive an unground piece of surgical foam, wherein the housing is configured to receive pulverized and shredded foam from a ground-up piece of the surgical foam and wherein the housing is also configured to allow removal of pulverized and shredded foam for use of the pulverized and shredded foam by a surgeon;
at least one shaft having a plurality of teeth attached thereto, wherein the plurality of teeth are spaced, located, and configured such that rotation of the plurality of teeth causes a piece of surgical foam in contact therewith to be pulled into the housing, while being pulverized and shredded; and
an actuator operably connected to the plurality of teeth and configured to rotate the plurality of teeth;
and
a sheet of surgical foam.

14. A method of embolization, the method comprising:
inserting a sheet of surgical foam into an apparatus configured to receive the sheet;
actuating an actuator operably connected to a plurality of teeth so as to rotate the plurality of teeth until the sheet is pulverized and/or shredded; and
injecting the pulverized and/or shredded foam into a blood vessel to form an embolus in the blood vessel.

15. The method of claim 14, further comprising removing pulverized and shredded foam from the apparatus and placing it in a syringe.

16. The method of claim 15, further comprising suspending at least partially the pulverized and shredded foam in saline and/or a contrast agent.

17. The method of claim 14, further comprising utilizing the apparatus during an embolization procedure.

18. The method of claim 17, further comprising disposing of the apparatus after the embolization procedure is completed.

19. The method of claim 14, wherein the plurality of teeth are spaced, located, and configured such that rotation of the plurality of teeth causes the sheet of surgical foam in contact therewith to be pulled into a housing, while being pulverized and shredded.

20. The method of claim 14, wherein the apparatus further comprises a housing configured to receive pulverized and shredded foam and also configured to allow removal of pulverized and shredded foam for use of the pulverized and shredded foam by a surgeon.
